(19) United States
(12) Patent Application Publication
(10) Pub. No.: US 2014/0240673 A1
(43) Pub. Date: Aug. 28, 2014

(54) IMAGE PROCESSING APPARATUS, OPHTHALMOLOGIC IMAGING APPARATUS, OPHTHALMOLOGIC IMAGING SYSTEM, OPHTHALMOLOGIC IMAGING METHOD, AND NON-TRANSITORY TANGIBLE MEDIUM HAVING PROGRAM STORED THEREON

(71) Applicant: CANON KABUSHIKI KAISHA, Tokyo (JP)

(72) Inventor: Tomoyuki Iwanaga, Yokohama-shi (JP)

(73) Assignee: CANON KABUSHIKI KAISHA, Tokyo (JP)

(21) Appl. No.: 14/178,640

(22) Filed: Feb. 12, 2014

(30) Foreign Application Priority Data
Feb. 25, 2013 (JP) 2013-034838

(51) Int. Cl. A61B 3/12; A61B 3/15

(52) U.S. Cl. CPC A61B 3/12; A61B 3/15
USPC 351/207; 351/206

(57) ABSTRACT

Provided is an ophthalmologic imaging apparatus for analyzing a fundus image of an eye to be inspected, including: an acquiring unit for acquiring a plurality of autofluorescence images obtained by imaging the eye to be inspected at different times; a generation unit for generating a plurality of fluorescence brightness images obtained by adjusting brightness of the plurality of autofluorescence images; and a display control unit for controlling a display unit to display the plurality of autofluorescence images and the plurality of fluorescence brightness images side by side for each time when the eye to be inspected is imaged.

### BACKGROUND OF THE INVENTION

1. Field of the Invention

The present invention relates to an image processing apparatus for analyzing a fundus image of an eye to be inspected, an ophthalmologic imaging apparatus including the image processing apparatus, an ophthalmologic imaging system, an ophthalmologic imaging method, and a non-transitory tangible medium having stored thereon a program for operating the apparatus or executing the method.

2. Description of the Related Art

There is known an ophthalmologic imaging apparatus, which irradiates a fundus of an eye to be inspected with light having a selected wavelength and uses a filter for filtering autofluorescence emitted from the fundus so as to take an autofluorescence image. As to the autofluorescence image, it is known to use a filter that transmits infrared light so as to capture fluorescent light mainly from lipofuscin.

Japanese Patent Application Laid-Open No. 2010-279536 discloses a technology in autofluorescence imaging of the fundus, in which a reference value for pixel values in a specific region of a fundus autofluorescence image of the eye to be inspected is determined, and pixels having pixel values beyond the reference value are displayed in a color.

Japanese Patent Application Laid-Open No. 2010-274048 discloses a technology of using a fundus camera capable of autofluorescence imaging of the fundus, in which the fundus is illuminated with illumination light selected for autofluorescence, a fundus image is taken without using a filter, and a monochrome image is generated.

Japanese Patent Application Laid-Open No. 2006-334044 discloses a technology in fluorescence imaging of the fundus, in which an arbitrary point is designated on a fluorescence fundus image, and a region having a brightness value larger than or equal to a brightness value of the arbitrary point is extracted from the fluorescence fundus image.

### SUMMARY OF THE INVENTION

According to one embodiment of the present invention, there is provided an image processing apparatus including: an acquiring unit for acquiring a plurality of autofluorescence images obtained by imaging an eye to be inspected at different times; a generation unit for generating a plurality of fluorescence brightness images obtained by adjusting brightness of the plurality of autofluorescence images; and a display control unit for controlling a display unit to display the plurality of autofluorescence images and the plurality of fluorescence brightness images side by side for each time when the eye to be inspected is imaged.

Further, according to one embodiment of the present invention, there is provided an ophthalmologic imaging apparatus including: an illumination unit for illuminating a fundus of an eye to be inspected with illumination light; a second wavelength selection unit for selecting an autofluorescence excitation light from the illumination light; a wavelength selection unit for transmitting autofluorescence excited at the fundus by the autofluorescence excitation light illuminating the fundus and filtering fundus reflection light of the autofluorescence excitation light; an imaging unit for taking an autofluorescence image formed by the light filtered by the wavelength selection unit; and an image processing unit for generating a fluorescence brightness image as a fluorescence image having brightness corresponding to fluorescence light intensity and a fluorescence distribution image having emphasized contrast, from the fluorescence image taken by the imaging unit.

Further features of the present invention will become apparent from the following description of exemplary embodiments with reference to the attached drawings.

### BRIEF DESCRIPTION OF THE DRAWINGS

FIG. 1 is a schematic diagram illustrating an example of the entire structure according to an embodiment of the present invention.

FIGS. 2A and 2B are diagrams illustrating specific examples of position adjustment and focus adjustment, respectively.

FIGS. 3A, 3B and 3C are diagrams illustrating fluorescence images obtained in accordance with processes.

FIGS. 4A and 4B are graphs showing histograms indicating gradation distributions of the fluorescence images in accordance with processes.

FIGS. 5A and 5B are diagrams illustrating details of operation switch portions of other embodiments of the present invention.

FIG. 6 is a block diagram illustrating a fluorescence image generation system according to the embodiment of the present invention.

FIG. 7 is a flowchart illustrating a process that is performed for generating a fluorescence image.

### DESCRIPTION OF THE EMBODIMENTS

An autofluorescence image is acquired by imaging as a fluorescence image, for example, a distribution of brightness (intensity) in a fundus image, which varies in accordance with an amount of fluorescent material (such as lipofuscin). In general, an autofluorescence image of a young person is dark, and the autofluorescence image becomes brighter with age. In addition, the autofluorescence image shows various fluorescence distributions due to diseases. In the autofluorescence image, the brightness and distribution of autofluorescence are important information for diagnosis.

Here, there is a case where the fluorescence distribution is not clear because the autofluorescence image is dark depending on age or disease of a subject. In addition, there is considered a case where it becomes difficult to acquire images having the same fluorescence brightness for the same patient because of development of disease, for example. It is difficult to determine whether such an image variation is due to development of disease or due to photography conditions, or due to other causes. There is considered a case where useful information concerning the disease cannot be obtained based only on the image.

Therefore, it is an object of embodiments of the present invention to enable to display an autofluorescence image suitable for an eye to be inspected concerning brightness (intensity) of the autofluorescence image and a distribution thereof, so as to provide more information including temporal variation.

An image processing apparatus according to one embodiment of the present invention generates a plurality of fluorescence brightness images obtained by adjusting brightness (intensity) of a plurality of autofluorescence images obtained by imaging the eye to be inspected at different times, and displays on a display unit the plurality of autofluorescence images and the plurality of fluorescence brightness images side by side for each time when the eye to be inspected is imaged.

In addition, an image processing apparatus according to another embodiment of the present invention generates a plurality of fluorescence distribution images obtained by emphasizing contrast of a plurality of autofluorescence images obtained by imaging an eye to be inspected at different times, and displays on a display unit the plurality of autofluorescence images and the plurality of fluorescence distribution images side by side for each time when the eye to be inspected is imaged.

According to this embodiment, at least one of brightness (intensity) and contrast of the autofluorescence image is adjusted so that the autofluorescence image suitable for the eye to be inspected can be displayed. Thus, it is possible to provide more information including temporal variation.

In the following, embodiments of the present invention are described in detail with reference to the drawings.

Structure of Fundus Imaging Apparatus

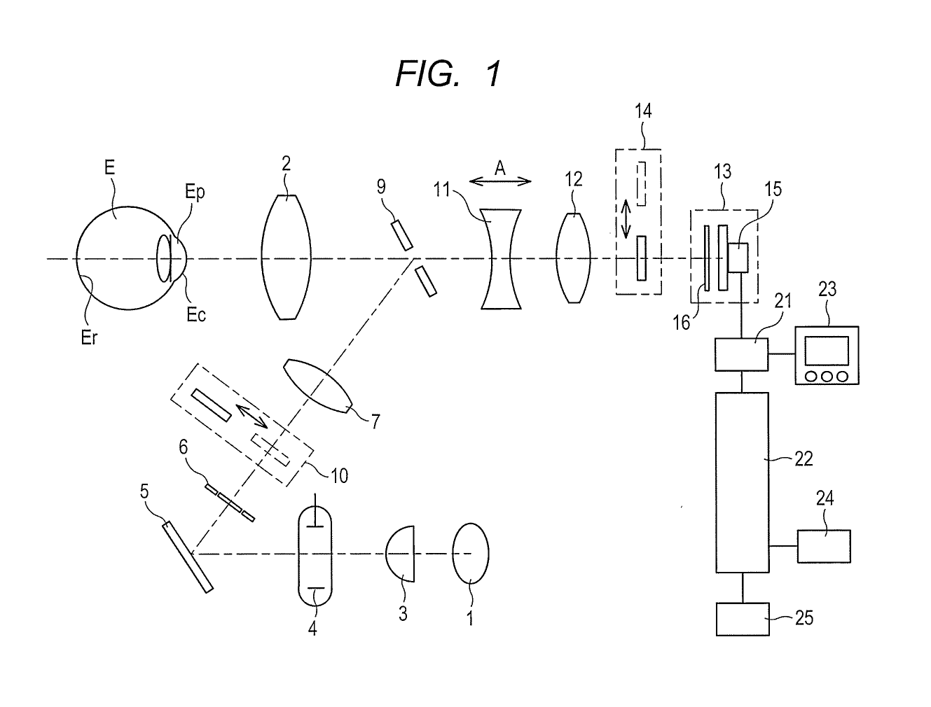

FIG. 1 is a structure diagram of an ophthalmologic imaging apparatus according to this embodiment. On an optical path from an observation light source 1 to an objective lens 2 in front of an eye to be inspected E, there are a condenser lens 3, an imaging light source 4, a cold mirror 5 as an example of an optical member that reflects visible light and transmits infrared light, a stop 6 having a ring-like aperture, a relay lens 7, and a perforated mirror 9, arranged in order. Further, between the stop 6 and the relay lens 7, an autofluorescence excitation filter 10 is disposed in an insertable and removable manner in the optical path. With these members, an illumination unit is constructed. The autofluorescence excitation filter 10 is inserted into an illumination optical path when performing autofluorescence observation and imaging, while the autofluorescence excitation filter 10 is removed from the illumination unit when performing color imaging, so as to function as a second wavelength selection unit in the present invention. Further, the above-mentioned optical member may be a dichroic mirror or any member that can selectively separate wavelengths, other than the cold mirror. In addition, it is possible to configure the above-mentioned optical member by a total reflection mirror and to dispose a visible light transmission filter that blocks infrared light and transmits visible light additionally in the illumination optical path.

On the optical path in the transmission direction of the perforated mirror 9, there are arranged a focusing lens 11, an imaging lens 12, and a color imaging unit 13. In addition, between the imaging lens 12 and the color imaging unit 13, there is disposed an autofluorescence filter 14 that blocks autofluorescence excitation light and transmits only fluorescence light in an insertable and removable manner in the optical path, and hence an observation and imaging optical system is constructed. The color imaging unit 13 includes an image pickup element 15, and a tricolor separation color filter 16 including a red color filter that transmits near infrared light. The autofluorescence filter 14 is inserted into the observation and imaging optical path when performing the autofluorescence imaging, while the autofluorescence filter is removed from the observation and imaging optical system when performing the color imaging, so as to function as a wavelength selection unit in the present invention.

An output of the image pickup element 15 is connected to a system control portion 22 via an image signal processing portion 21 as an image processing unit, namely a generation unit. In addition, the image signal processing portion 21 is connected to a display portion 23, on which an observation image of the eye to be inspected E is displayed for observing the eye to be inspected E. In addition, the system control portion 22 is connected to an image storing portion 24 as an image storing unit and an operation switch portion 25 so that a control system of the entire fundus camera is constructed. In addition, the image pickup element 15 for imaging or acquiring the fluorescence image and the accompanying structure correspond to an acquiring unit in the present invention.

When a color image is acquired, a light beam emitted from the observation light source 1 passes through the condenser lens 3 and the imaging light source 4 and is reflected by the mirror 5. The reflection light from the cold mirror 5 passes through the stop 6 and the relay lens 7, is reflected at the periphery of the perforated mirror 9, passes through the objective lens 2, and irradiates a fundus Er of the eye to be inspected E as visible light. In this case, the autofluorescence excitation filter 10 is removed from the illumination unit.

Reflection light from the fundus Er passes through the objective lens 2, the aperture of the perforated mirror 9, the focusing lens 11, and the imaging lens 12, and an image thereof is formed on the image pickup element 15. In this case, because the autofluorescence filter 14 is removed from the fundus observation and imaging optical system, the reflection light from the fundus Er can be observed as it is as a fundus image on the display portion 23.

An examiner operates the apparatus to move backward and forward, right and left, and up and down by using a position adjustment index and an operation portion (not shown) while viewing the fundus image, so as to perform position adjustment with respect to the eye to be inspected E. Further, the focusing lens 11 is moved by using a focus adjustment index so that focus adjustment is performed.

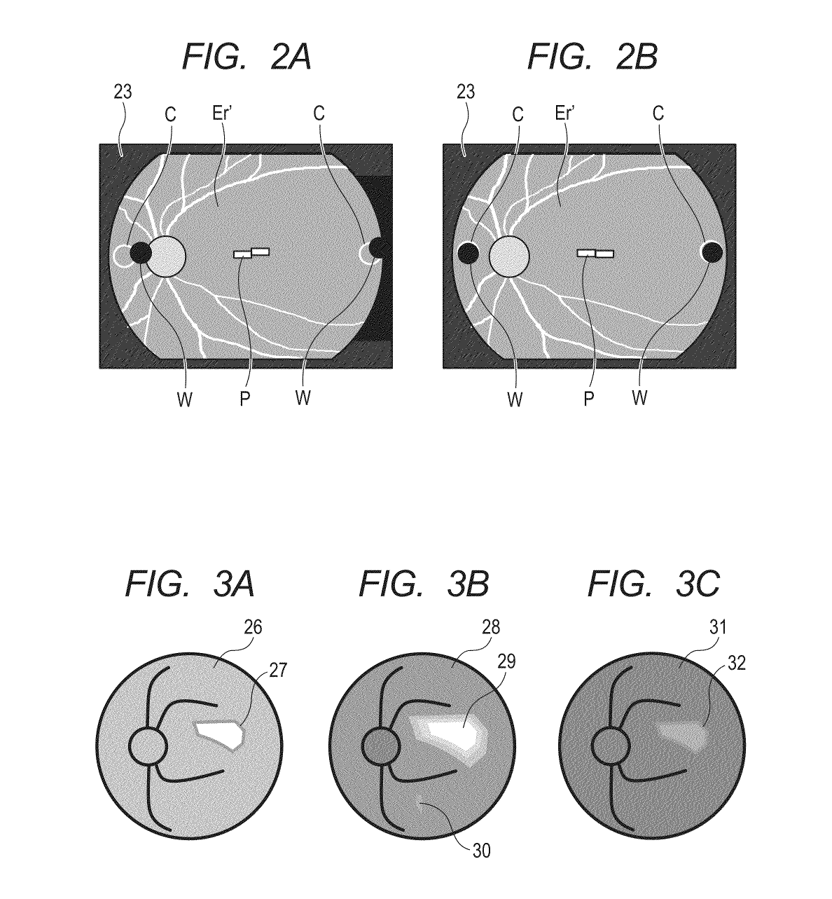

FIGS. 2A and 2B illustrate observed states of a fundus image Er′ on the display portion 23, and the fundus image Er′ is displayed in an enlarged manner for easy observation. FIG. 2A illustrates a state where the position adjustment and the focus adjustment are not completed, in which position adjustment circles C are shifted from position adjustment indexes W, and focus adjustment indexes P are also shifted. FIG. 2B illustrates a state where the position adjustment and the focus adjustment are completed, in which the position adjustment circles C are matched with the position adjustment indexes W, and the focus adjustment indexes P are aligned.

After the position adjustment and the focus adjustment of the fundus image Er′ are finished, the examiner presses an imaging switch in the operation switch portion 25 so that the system control portion 22 controls the imaging light source 4 to emit light. A light beam emitted from the imaging light source 4 passes along the same path as the light beam from the observation light source 1 and irradiates the fundus Er. Reflection light from the illuminated fundus Er forms an image on the image pickup element 15 similarly to the case of observation. Image data of the formed fundus image Er′ are stored as a color image in the image storing portion 24 via the image signal processing portion 21 and the system control portion 22, and the fundus image Er′ is displayed on the display portion 23.

When performing the autofluorescence imaging, the autofluorescence excitation filter 10 is inserted into the illumination optical path. The light beam emitted from the observation light source 1 passes through the condenser lens 3 and the imaging light source 4 and is reflected by the mirror 5. The reflection light from the cold mirror 5 passes through the stop 6 and the autofluorescence excitation filter 10 for wavelength selection, is reflected at the periphery of the perforated mirror 9, passes through the objective lens 2, and irradiates the fundus Er. The structure including the observation light source 1 and the autofluorescence excitation filter 10 for irradiating the fundus of the eye to be inspected with the autofluorescence excitation light functions as a fluorescence excitation light illumination unit in the present invention. Further, in this embodiment, as the structure for illuminating the fundus with the autofluorescence excitation light, there is exemplified the structure including the imaging light source 4 as a light source unit for emitting the illumination light for illuminating the fundus, and the autofluorescence excitation filter 10 as the second wavelength selection unit for selecting the autofluorescence excitation light from the illumination light emitted from the imaging light source 4. However, the present invention is not limited to this structure. As long as the fundus is illuminated with the autofluorescence excitation light, it is possible to replace this structure with, for example, a structure that directly emits the autofluorescence excitation light. Therefore, in the present invention, the structure is generically referred to as an autofluorescence excitation light illumination unit.

The reflection light from the illuminated fundus Er passes through a pupil Ep, the objective lens 2, the aperture of the perforated mirror 9, the focusing lens 11, and the imaging lens 12, and an image thereof is formed on the image pickup element 15. In this case, because the autofluorescence filter 14 is removed from the fundus observation and imaging optical system, the reflection light from the fundus Er, which is obtained by wavelength light which passes through the autofluorescence excitation filter 10 for the wavelength selection, can be observed as a fundus image Er′.

The examiner performs the position adjustment of the apparatus with respect to the eye to be inspected E by using the position adjustment index W and performs the focus adjustment by using the focus adjustment index P while viewing the fundus image Er′, in the same manner as described above with reference to FIGS. 2A and 2B.

After the position adjustment and the focus adjustment of the fundus image Er′ are finished, the examiner presses the imaging switch in the operation switch portion 25 so that the system control portion 22 inserts the autofluorescence filter 14 into the fundus observation and imaging optical system and controls the imaging light source 4 to emit light. The light beam emitted from the imaging light source 4 passes along the same optical path as the light beam from the observation light source 1 so as to irradiate the fundus Er with the autofluorescence excitation light as the wavelength light after passing through the autofluorescence excitation filter 10. In the illuminated fundus Er, the light having the wavelength selected by the autofluorescence excitation filter 10 excites the fluorescent material to emit the fluorescence light.

Fluorescence light and illumination reflection light or fundus reflection light from the fundus Er passes through the pupil Ep, the objective lens 2, the aperture of the perforated mirror 9, the focusing lens 11, and the imaging lens 12, and the autofluorescence filter 14 blocks the wavelength light after passing through the autofluorescence excitation filter 10. In this way, only the fluorescence light from the fundus image Er′ passes through and forms its image on the image pickup element 15.

The formed fundus image Er′ is made monochrome by the image signal processing portion 21, and is stored in the image storing portion 24 via the system control portion 22, as a first autofluorescence image 26 that is a monochrome image as illustrated in FIG. 3A.

Process Performed on Autofluorescence Image

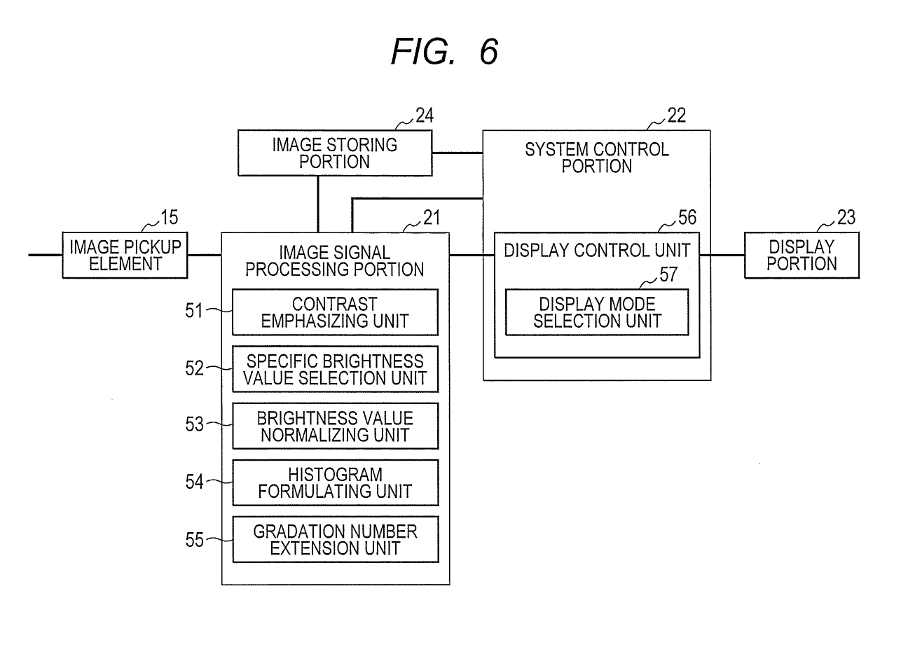

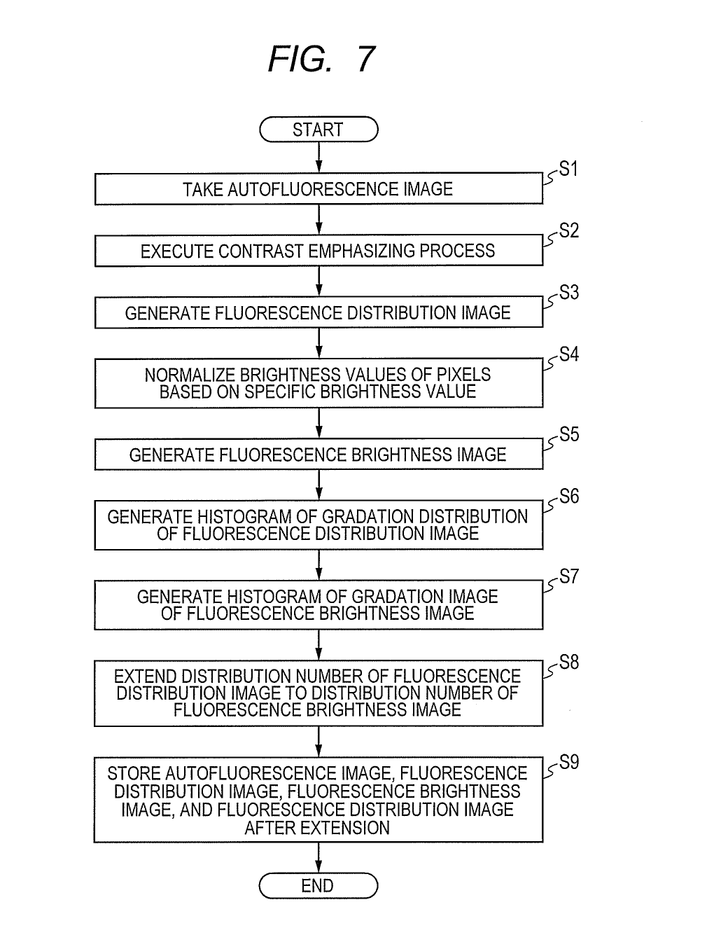

Next, a process performed on the obtained first autofluorescence image 26 is described in detail with reference to FIG. 6 illustrating a block diagram of a fluorescence image generation system according to an embodiment of the present invention, and FIG. 7 illustrating a flowchart of a process performed for generating the fluorescence image. The following process is performed by the system control portion 22, which serves as a generation unit in the present invention. The system control portion 22 performs the process of emphasizing contrast of the first autofluorescence image 26 stored in the image storing portion 24 (that has been taken or acquired in Step S1) using a contrast emphasizing unit 51 included in the image signal processing portion 21 (Step S2). By this process, an autofluorescence distribution image (fluorescence distribution image) 28 illustrated in FIG. 3B, in which the distribution of autofluorescence can be easily recognized, is generated in Step S3. Next, or in parallel with Step S3, a specific brightness value selection unit 52 selects a specific brightness value from the image storing portion 24, and a brightness value normalizing unit 53 normalizes brightness values of individual pixels to the specific brightness value based on the selected brightness value (Step S4). Brightness of the autofluorescence image is adjusted based on pixel values after the normalization, and an autofluorescence brightness image (fluorescence brightness image) 31 corresponding to actual fluorescence light intensity is generated (Step S5). The autofluorescence brightness image normalized by the generated predetermined brightness value is illustrated in FIG. 3C. A region of the system control portion 22, which functions as a display control unit 56 for indicating an image to be displayed on the display portion 23 as the display unit, selects an image to be displayed from among the images and controls the display portion 23 to display the image.

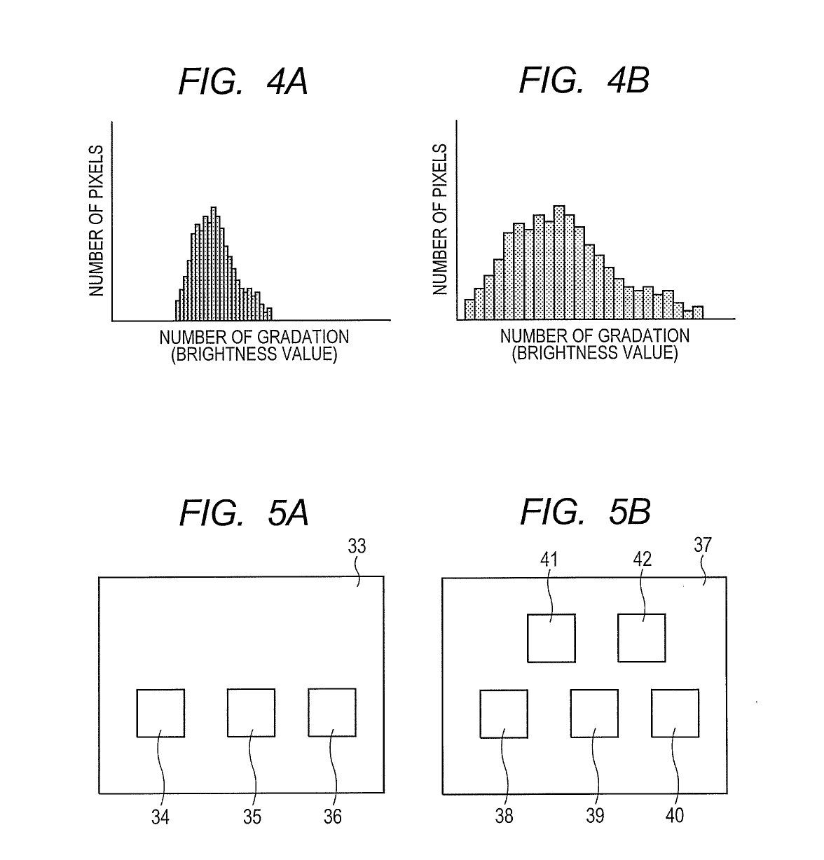

Next, a histogram generation unit 54 generates a histogram of gradation distribution of the autofluorescence distribution image 28 (Step S6). FIG. 4A shows a histogram of gradation distribution of the first autofluorescence image 26 illustrated in FIG. 3A.

In addition, the histogram generation unit 54 also generates a histogram of gradation distribution of the autofluorescence brightness image 31 (Step S7). FIG. 4B shows a histogram of gradation distribution of the autofluorescence distribution image illustrated in FIG. 3B.

In the image signal processing portion 21, a gradation number expansion unit 55 increases the gradation number so that the histogram of FIG. 4A becomes the histogram of FIG. 4B (Step S8), and the autofluorescence distribution image illustrated in FIG. 3B is generated. In this way, the brightness distribution of an autofluorescence portion 29 of FIG. 3B can be viewed in detail, and an autofluorescence portion 30 that has low brightness and hence is hardly checked in FIG. 3A can be checked. As to the low brightness mentioned here, there is considered a case where a brightness value of a so-called background is 10 and a brightness value of a fluorescence portion is 20.

Here, the image storing portion 24 as the storing unit temporarily stores the first autofluorescence image 26, and the image signal processing portion 21 generates the autofluorescence distribution image 28 and the autofluorescence brightness image 31. As an image that is permanently stored in the image storing portion 24, the first autofluorescence image 26 may be stored. Alternatively, the first autofluorescence image 26 may be erased, and the generated autofluorescence distribution image 28 and the generated autofluorescence brightness image 31 may be stored. Alternatively, as shown in Step S9 of the flowchart, all of the images in this stage may be stored.

Further, in a case where a so-called personal computer (PC) is used, it is considered that the structure related to the display portion (monitor) 23 of the ophthalmologic imaging apparatus relatively easily includes the above-mentioned display control portion. However, as exemplified in this embodiment, it is possible that the display portion is used as an external device and the display control unit 56 included in the system control portion 22 instructs the image to be displayed. In addition, it is preferred that the display control unit 56 include a display mode selection unit 57 described later in another embodiment.

According to this embodiment, the display portion 23 can display at least one of the fluorescence image, the fluorescence brightness image, and the fluorescence distribution image, and the display control unit instructs which one of images is to be displayed. In accordance with an external instruction, the display control unit controls the display portion 23 to display at least one of the fluorescence image, the fluorescence brightness image, and the fluorescence distribution image of the eye to be inspected stored in the storing unit, and at least one of the newly acquired fluorescence image, fluorescence brightness image, and fluorescence distribution image, in an associated manner. In this embodiment, this operation is achieved by the structure including the storing unit for storing the fluorescence distribution image and the fluorescence brightness image generated by the image processing unit.

In this embodiment, with this structure, the fluorescence distribution image or the fluorescence brightness image of the eye to be inspected acquired in the past can be viewed without using the image processing unit, and therefore time for the display can be shortened. In addition, the images stored in advance and the newly acquired images are displayed in an associated manner. For instance, there is a case where a fluorescence degree is changed because a condition of a disease is developed with time. Also in this case, it is possible to grasp a state of a lesion securely and easily by displaying the past fluorescence brightness image indicating brightness corresponding to actual fluorescence light intensity based on the specific reference value, or the part fluorescence distribution image indicating contrast in more detail with the increased gradation number for easy visual recognition, and the current image corresponding to the past image in an associated manner.

In addition, according to this embodiment, the fluorescence image acquired by the acquiring unit is stored as the first fluorescence image, and at least one of the fluorescence brightness image and the fluorescence distribution image is generated from the first fluorescence image. Thus, it is possible to provide the autofluorescence image corresponding to necessity, thereby preventing an increase of capacity of the storing unit more than necessity.

Change of Imaging Mode

Next, an example of a change pattern of imaging modes in this embodiment is described. Here, in addition to the ophthalmologic imaging apparatus described above, an operation switch portion 33 is disposed instead of the operation switch portion 25. The operation switch portion 33 is equipped with an imaging switch 34, an imaging mode selection switch 35, and a display mode selection switch 36 as illustrated in FIG. 5A.

The imaging mode selection switch 35 is preferably included in the system control portion 22 including the operation switch portion 33, and is more preferably included in the display control portion 56. The imaging mode selection switch 35 can select an imaging mode such as a color imaging mode or an autofluorescence imaging mode. The display mode selection switch 36 is enabled in the autofluorescence imaging mode so as to select a fluorescence distribution display mode and a fluorescence brightness image display mode.

There is described a case where the autofluorescence imaging mode is selected by the imaging mode selection switch 35, and the fluorescence distribution display mode is selected by the display mode selection switch 36. When the examiner presses the imaging switch 34 in the operation switch portion 33 after the position adjustment and the focus adjustment of the fundus image Er′ are finished, the system control portion 22 inserts the autofluorescence filter 14 into the fundus observation and imaging optical system and controls the imaging light source 4 to emit light. The light beam emitted from the imaging light source 4 passes along the same optical path as the light beam from the observation light source 1 so as to irradiate the fundus Er with the wavelength light after passing through the autofluorescence excitation filter 10. In the illuminated fundus Er, the light having the wavelength selected by the autofluorescence excitation filter 10 excites the fluorescent material to emit the fluorescence light.

Fluorescence light and illumination reflection light or fundus reflection light from the fundus Er passes through the pupil Ep, the objective lens 2, the aperture of the perforated mirror 9, the focusing lens 11, and the imaging lens 12, and the autofluorescence filter 14 blocks the wavelength light after passing through the autofluorescence excitation filter 10. In this way, only the fluorescence light from the fundus image Er′ passes through and forms its image on the image pickup element 15.

The formed fundus image Er′ is made monochrome by the image signal processing portion 21, and is stored in the image storing portion 24 via the system control portion 22, as the first autofluorescence image 26 as illustrated in FIG. 3A.

The system control portion 22 controls the image signal processing portion 21 to emphasize the contrast of the first autofluorescence image 26 of FIG. 3A stored in the image storing portion 24 so as to generate the autofluorescence distribution image 28 illustrated in FIG. 3B, in which the autofluorescence distribution can be easily recognized, and the system control portion 22 further controls the display portion 23 to display the autofluorescence distribution image 28.

Next, there is described a case where the autofluorescence imaging mode is selected by the imaging mode selection switch 35, and the fluorescence brightness image display mode is selected by the display mode selection switch 36. When the examiner presses the imaging switch 34 in the operation switch portion 33 after the position adjustment and the focus adjustment of the fundus image Er′ are finished, the first autofluorescence image 26 as illustrated in FIG. 3A is stored in the image storing portion 24 in the same manner as in the case where the fluorescence distribution display mode is selected by the display mode selection switch 36.

The system control portion 22 controls the image signal processing portion 21 to normalize the first autofluorescence image 26 stored in the image storing portion 24 by the specific brightness value so as to generate the autofluorescence brightness image 31. The display control unit described above controls the display portion 23 to display the autofluorescence brightness image 31.

When the imaging switch 34 is pressed, the system control portion 22 stores the first autofluorescence image in the image storing portion 24. Further, in accordance with the display mode selected by the display mode selection switch 36, the system control portion 22 generates the autofluorescence distribution image 28 in which contrast of the image to be displayed is emphasized by the image signal processing portion 21 so that the autofluorescence distribution can be easily recognized, or the autofluorescence brightness image 31 obtained by normalizing the image to be displayed by the specific brightness value, and the system control portion 22 further controls the display portion 23 to display the generated image. The display mode selection switch 36 serves as a part of the display mode selection unit.

Here, the first autofluorescence image 26 is temporarily stored in the image storing portion 24, and the image signal processing portion 21 generates the autofluorescence distribution image 28 and the autofluorescence brightness image 31. As the image stored permanently in the image storing portion 24, the first autofluorescence image 26 may be stored. Alternatively, the first autofluorescence image 26 may be erased, and the generated autofluorescence distribution image 28 and autofluorescence brightness image 31 may be stored. Alternatively, all of the images may be stored.

According to this embodiment, when the fluorescence distribution display mode is selected by the display mode selection unit, at least the fluorescence distribution image is displayed. When the fluorescence brightness image display mode is selected, at least the fluorescence brightness image is displayed. Thus, it is possible to provide the fluorescence image suitable for the purpose of examination.

Display of Progress Information

Here, there is described a display form that enables to inform progress of a lesion or the like in the eye to be inspected. As illustrated in FIG. 5B, an operation switch portion 37 is disposed instead of the operation switch portion 25, and the operation switch portion 37 is equipped with an imaging switch 38, an imaging mode selection switch 39, a display mode selection switch 40, a progress observation switch 41, and an image selection switch 42. In addition, the image storing portion 24 stores the images and information of the eye to be inspected as associated information. In more detail, the image pickup element 15 acquires the autofluorescence image of the specific eye to be inspected at different times, for example, at each of a plurality of times when the eye to be inspected is examined.

The system control portion 22 controls the imaging mode selection switch 39 to select the autofluorescence imaging mode and controls the display mode selection switch 40 to select the fluorescence distribution display mode. When the progress observation switch 41 is turned on and the imaging switch 38 is pressed, the image storing portion 24 stores the first autofluorescence image as illustrated in FIG. 3A. Then, the first autofluorescence image stored in the image storing portion 24 is processed by the image signal processing portion 21 so that the autofluorescence distribution image 28 is generated, and the past autofluorescence distribution image of the same eye to be inspected stored in the image storing portion 24 is retrieved. The autofluorescence distribution image just after the imaging and a plurality of the past autofluorescence distribution images are displayed on the display portion 23. In other words, the system control portion 22 as the display control portion controls the display portion 23 to display the plurality of autofluorescence images, the plurality of fluorescence brightness images, the plurality of fluorescence distribution images, and a more arbitrary image side by side simultaneously for each time when the eye to be inspected is imaged.

In addition, the system control portion 22 controls the imaging mode selection switch 39 to select the autofluorescence imaging mode and controls the display mode selection switch 40 to select the fluorescence brightness image display mode. When the progress observation switch 41 is turned on and the imaging switch 38 is pressed, the image storing portion 24 stores the first autofluorescence image 26 as illustrated in FIG. 3A. Then, the first autofluorescence image 26 stored in the image storing portion 24 is processed by the image signal processing portion 21 so that the autofluorescence brightness image 31 is generated, and the past autofluorescence brightness image 31 of the same eye to be inspected stored in the image storing portion 24 is retrieved. The autofluorescence brightness image just after the imaging and a plurality of the past autofluorescence brightness images are displayed on the display portion 23.

In other words, in this embodiment, the display control unit includes the progress observation switch 41 serving as a part of a progress observation selection unit. When the progress observation mode is selected by the progress observation selection unit, the display control unit controls the display portion 23 to display simultaneously at least the image corresponding to the mode selected by the display mode selection unit, and a second fluorescence image that is an image based on the first fluorescence image of the image of the eye to be inspected stored in the storing unit in advance, which corresponds to a mode selected by the display mode selection unit.

Here, when the progress observation switch 41 is turned on and the fluorescence distribution display mode is selected by the display mode selection switch 40, a plurality of sequential autofluorescence distribution images of the same eye to be inspected are displayed on the display portion 23. In addition, when the fluorescence brightness display mode is selected by the display mode selection switch 40, a plurality of sequential autofluorescence brightness images of the same eye to be inspected are displayed on the display portion 23. However, when the fluorescence distribution display mode is selected by the display mode selection switch 40, at least one autofluorescence brightness image is displayed on the display portion 23 together with the plurality of sequential autofluorescence distribution images of the same eye to be inspected. Then, when the fluorescence brightness display mode is selected by the display mode selection switch 40, at least one autofluorescence distribution image may be displayed on the display portion together with the plurality of sequential autofluorescence brightness images of the same eye to be inspected.

Note that, the present invention is not limited to the embodiment described above, and the acquiring unit may acquire the autofluorescence image obtained by imaging the eye to be inspected by an external ophthalmologic imaging apparatus. In addition, including the external ophthalmologic imaging apparatus, the ophthalmologic imaging apparatus may be not only the fundus camera but also an SLO apparatus capable of autofluorescence imaging.

According to the present invention, when the progress observation is selected by the progress observation selection unit and the fluorescence distribution image display mode is selected by the display mode selection unit, the plurality of fluorescence distribution images are displayed. When the fluorescence brightness image display mode is selected by the display mode selection unit, the plurality of fluorescence brightness images are displayed. In this way, a time-sequential change of fluorescence distribution images or fluorescence brightness images can be checked.

In addition, according to the present invention, when the progress observation is selected by the progress observation selection unit and the fluorescence brightness image display mode is selected by the display mode selection unit, the fluorescence distribution image is displayed together with the plurality of fluorescence brightness images. Thus, the fluorescence distribution and the time-sequential change of fluorescence brightness images can be checked simultaneously.

### Other Embodiments

Note that, the present invention is also implemented by executing the following process. Specifically, in this process, software (program) for implementing the functions of the above-mentioned embodiment is supplied to a system or an apparatus via a network or various kinds of storage medium, and a computer (or CPU, MPU, etc.) of the system or the apparatus reads out and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-034838, filed Feb. 25, 2013, which is hereby incorporated by reference herein in its entirety.

What is claimed is:

1. An image processing apparatus, comprising:
an acquiring unit for acquiring a plurality of autofluorescence images obtained by imaging an eye to be inspected at different times;
a generation unit for generating a plurality of fluorescence brightness images obtained by adjusting brightness of the plurality of autofluorescence images; and
a display control unit for controlling a display unit to display the plurality of autofluorescence images and the plurality of fluorescence brightness images side by side for each time when the eye to be inspected is imaged.

2. An image processing apparatus according to claim 1, wherein the generation unit generates a plurality of fluorescence distribution images obtained by emphasizing contrast of the plurality of autofluorescence images.

3. An image processing apparatus according to claim 2, wherein the display control unit controls the display unit to display the plurality of autofluorescence images, the plurality of fluorescence brightness images, and the plurality of fluorescence distribution images, side by side for each time when the eye to be inspected is imaged.

4. An image processing apparatus, comprising:
an acquiring unit for acquiring a plurality of autofluorescence images obtained by imaging an eye to be inspected at different times;
a generation unit for generating a plurality of fluorescence distribution images obtained by emphasizing contrast of the plurality of autofluorescence images; and
a display control unit for controlling a display unit to display the plurality of autofluorescence images and the plurality of fluorescence distribution images side by side for each time when the eye to be inspected is imaged.

5. An image processing apparatus, comprising:
an acquiring unit for acquiring a plurality of autofluorescence images obtained by imaging an eye to be inspected at different times;
a generation unit for generating a plurality of fluorescence brightness images obtained by adjusting brightness of the plurality of autofluorescence images and generating a plurality of fluorescence distribution images obtained by emphasizing contrast of the plurality of autofluorescence images;
a display mode selection unit for selecting one of a plurality of display modes including a fluorescence brightness image display mode for displaying the plurality of fluorescence brightness images and a fluorescence distribution image display mode for displaying the plurality of fluorescence distribution images; and
a display control unit for controlling a display unit to display the plurality of images of the display mode selected by the display mode selection unit and the plurality of autofluorescence images side by side for each time when the eye to be inspected is imaged.

6. A non-transitory tangible medium having stored thereon a program for causing a computer to execute functions of the image processing apparatus according to claim 1.

7. An ophthalmologic imaging apparatus, comprising:
an autofluorescence excitation light illumination unit for illuminating a fundus of an eye to be inspected with autofluorescence excitation light;
a wavelength selection unit for transmitting autofluorescence excited at the fundus by the autofluorescence excitation light illuminating the fundus and filtering fundus reflection light of the autofluorescence excitation light;
an acquiring unit for acquiring an autofluorescence image formed by the light filtered by the wavelength selection unit; and
a generation unit for generating a fluorescence brightness image that is a fluorescence image having brightness corresponding to fluorescence light intensity in the autofluorescence image, and a fluorescence distribution image having emphasized contrast, from the autofluorescence image acquired by the acquiring unit.

8. An ophthalmologic imaging apparatus according to claim 7, wherein the autofluorescence excitation light illumination unit comprises a light source unit for emitting illumination light for illuminating the fundus of the eye to be inspected, and a second wavelength selection unit for selecting the autofluorescence excitation light from the illumination light emitted from the light source unit.

9. An ophthalmologic imaging apparatus according to claim 7,
wherein the generation unit comprises:
a contrast emphasizing unit for emphasizing contrast of the autofluorescence image so as to generate the fluorescence distribution image;
a specific brightness value selection unit for selecting a specific brightness value; and
a brightness value normalizing unit for normalizing brightness values of pixels generating the autofluorescence image based on the specific brightness value selected by the specific brightness value selection unit, and
wherein the generation unit generates the fluorescence brightness image based on the brightness value normalized by the brightness value normalizing unit.

10. An ophthalmologic imaging apparatus according to claim 7, further comprising:
a storing unit for storing the fluorescence distribution image and the fluorescence brightness image generated by the generation unit; and
a display control unit for controlling the display unit to display at least one of the autofluorescence image, the fluorescence brightness image, and the fluorescence distribution image,
wherein the display control unit controls, in accordance with an external instruction, the display unit to display the at least one of the autofluorescence image, the fluorescence brightness image, and the fluorescence distribution image of the eye to be inspected stored in the storing unit in advance, and at least one of a newly acquired autofluorescence image, fluorescence brightness image, and fluorescence distribution image, in an associated manner.

11. An ophthalmologic imaging apparatus according to claim 10,
wherein the display control unit includes a display mode selection unit for selecting a display mode of an image to be displayed on the display unit,
wherein the display mode selection unit is configured to select between a fluorescence distribution display mode for displaying at least the fluorescence distribution image and a fluorescence brightness display mode for displaying at least the fluorescence brightness image, and
wherein the external instruction is issued through the display mode selection unit.

12. An ophthalmologic imaging apparatus, comprising:
an illumination unit for illuminating a fundus of an eye to be inspected with illumination light;
a second wavelength selection unit disposed in the illumination unit, for selecting autofluorescence excitation light;
a wavelength selection unit for transmitting autofluorescence excited by the autofluorescence excitation light selected by the wavelength selection unit and filtering fundus reflection light of the autofluorescence excitation light;
an acquiring unit for acquiring an autofluorescence image formed by the light filtered by the wavelength selection unit;
a storing unit for storing the autofluorescence image acquired by the acquiring unit as a first fluorescence image; and
a generation unit for generating at least one of a fluorescence brightness image as a fluorescence image having brightness corresponding to fluorescence light intensity or a fluorescence distribution image having emphasized contrast from the first fluorescence image.

13. An ophthalmologic imaging apparatus according to claim 12, further comprising:
a display unit for displaying one of the fluorescence distribution image and the fluorescence brightness image; and
a display control unit for instructing the display unit to display the image,
wherein the display control unit includes a display mode selection unit for selecting any one of display modes to be executed on the display unit, the display modes including a fluorescence distribution display mode for displaying at least the fluorescence distribution image and a fluorescence brightness display mode for displaying at least the fluorescence brightness image.

14. An ophthalmologic imaging apparatus according to claim 13,
wherein the display unit is configured to simultaneously display a plurality of images,
wherein the display control unit further comprises a progress observation selection unit, and
wherein when the progress observation selection unit selects a progress observation mode, the display control unit controls the display unit to simultaneously display at least an image corresponding to a mode selected by the display mode selection unit, and a second fluorescence image that is an image based on the first fluorescence image of the eye to be inspected stored in the storing unit in advance and corresponding to a mode selected by the display mode selection unit.

15. An ophthalmologic imaging apparatus according to claim 14, wherein when the progress observation mode is selected, the display control unit controls the display unit to simultaneously display at least one image corresponding to a mode that is not selected by the display mode selection unit in addition to the second fluorescence image corresponding to the mode selected by the display mode selection unit.

16. An ophthalmologic imaging apparatus according to claim 15, wherein when the fluorescence brightness image display mode is selected by the display mode selection unit, the display control unit controls the display unit to display at least one fluorescence distribution image together with a plurality of the fluorescence brightness images.

17. An ophthalmologic imaging system, comprising:
an autofluorescence excitation light illumination unit for illuminating a fundus of an eye to be inspected with autofluorescence excitation light;
an autofluorescence filter for transmitting autofluorescence excited by the autofluorescence excitation light and filtering fundus reflection light of the autofluorescence excitation light;
an acquiring unit for acquiring an autofluorescence image formed by the light filtered by the autofluorescence filter; and
a generation unit for generating a fluorescence brightness image as a fluorescence image having brightness corresponding to fluorescence light intensity and a fluorescence distribution image having emphasized contrast, from the autofluorescence image acquired by the acquiring unit.

18. An ophthalmologic imaging system according to claim 17, wherein the autofluorescence excitation light illumination unit comprises a light source unit for emitting illumination light illuminating the fundus of the eye to be inspected, and a wavelength selection unit for selecting the autofluorescence excitation light from the illumination light emitted from the light source unit.

19. An ophthalmologic imaging method, comprising:
illuminating a fundus of an eye to be inspected with autofluorescence excitation light;
transmitting autofluorescence excited by the autofluorescence excitation light and filtering fundus reflection light of the autofluorescence excitation light;
acquiring an autofluorescence image formed by the light filtered in the filtering; and
generating a fluorescence brightness image as a fluorescence image having brightness corresponding to fluorescence light intensity and a fluorescence distribution image having emphasized contrast, from the autofluorescence image acquired in the acquiring.

20. A non-transitory tangible medium having stored thereon a program for causing a computer to execute steps of the ophthalmologic imaging method according to claim 19.
